(19) United States
(12) Patent Application Publication
(10) Pub. No.: US 2026/0301892 A1
(43) Pub. Date: Oct. 1, 2026

(54) EXPERT PANEL SUPPORT SYSTEM AND EXPERT PANEL SUPPORT METHOD

(71) Applicant: HITACHI HIGH-TECH CORPORATION, Tokyo (JP)

(72) Inventors: Eichi WATABE, Tokyo (JP); Eiji KANAMORI, Tokyo (JP); Aya KURIKI, Tokyo (JP); Koichiro KIMOTSUKI, Tokyo (JP)

(73) Assignee: HITACHI HIGH-TECH CORPORATION, Tokyo (JP)

(21) Appl. No.: 19/573,420

(22) Filed: Mar. 20, 2026

(30) Foreign Application Priority Data
Mar. 27, 2025 (JP) 2025-054173

(51) Int. Cl. G16H 10/40; G16B 20/20

(52) U.S. Cl. CPC G16H 10/40; G16B 20/20

(57) ABSTRACT

An expert panel support system includes an acquisition processing unit configured to acquire interpretation result data that is data of an interpretation result for each gene variant of a first patient subjected to a gene panel test, and a display processing unit configured to display, based on the interpretation result data, a result of a test performed on the first patient for each gene variant in a list on a terminal used by a participant of an expert panel. Based on the interpretation result data, the display processing unit displays interpretation results of a gene variant of a plurality of second patients, who are patients different from the first patient and previously subjected to the gene panel test before the gene panel test performed on the first patient, on the terminal in association with the interpretation result of the gene variant of the first patient.

### BACKGROUND OF THE INVENTION

### 1. Field of the Invention

The present invention relates to a technique of an expert panel support system and an expert panel support method.

### 2. Description of Related Art

Cancer genome medical care has been often performed. In cancer genome medical care, a cancer gene panel test capable of detecting several tens to several hundreds of genes at once is performed for one patient. A change in a cancer-related gene is confirmed by a test result of the cancer gene panel test.

An expert panel, which is a meeting for performing medical interpretation based on a result of such a cancer gene panel test and studying an optimal treatment policy, is held. In the expert panel, an expert such as a doctor specializing in cancer medicine therapy or a doctor specializing in genetic medicine studies a treatment policy based on a test result of the cancer gene panel test.

PTL 1 discloses a method of controlling an information processing system and an information processing system for managing a result of a gene panel test by "a method for managing a test request of a gene panel test by a computer, the method including acquiring, for each of a plurality of test requests, information on the test request and an attribute of a test result in the gene panel test, and outputting display information for displaying each of the plurality of test requests and the attribute in association with each other".

### CITATION LIST

### PATENT LITERATURE

PTL 1: JP2024-074878A

### SUMMARY OF THE INVENTION

In reports created by test companies, additional information and pathogenic interpretation of gene variants are often insufficient, and doctors perform an expert panel preliminary study. The expert panel preliminary study refers to that members of an expert panel examine and study a detected gene variant in advance before the expert panel is held.

The number of gene panel tests and the number of gene variants found for each test tend to increase. Therefore, a load on the study of a gene panel test is expected to increase in the future.

In addition, there are many hospitals in which reports of test companies and information of an expert panel preliminary study are not systematically integrated, and it is difficult to efficiently refer to the information. Further, information on the expert panel preliminary study and information on a final determination by the expert panel are not sufficiently stored in a database and cannot be effectively utilized.

In particular, in the present circumstance, means for a member of an expert panel to refer to a study result (interpretation result) of a previous expert panel is limited, and there is a problem in efficiency.

In the technique disclosed in PTL 1, a previous interpretation result of an expert panel is not known, which is not convenient for a member of an expert panel.

The invention has been made in view of such a background, and an object of the invention is to provide a user-friendly display for a member of an expert panel.

In order to solve the above-described problem, the expert panel support system includes: an acquisition unit configured to acquire interpretation result data that is data of an interpretation result for each gene variant of a first patient subjected to a gene panel test; and a display processing unit configured to display, based on the interpretation result data, a result of a test performed on the first patient for each gene variant on a terminal used by a participant of an expert panel, in which the display processing unit displays, based on the interpretation result data, interpretation results of a gene variant of a plurality of second patients, who are patients different from the first patient and previously subjected to the gene panel test before the gene panel test performed on the first patient, on the terminal in association with the interpretation result of the gene variant of the first patient.

Other solutions will be described as appropriate in the embodiments.

According to the invention, it is possible to provide a user-friendly display for a member of an expert panel.

### BRIEF DESCRIPTION OF THE DRAWINGS

FIG. 1 is a diagram showing a configuration of an expert panel support system according to the present embodiment.

FIG. 2 is a diagram showing a hardware structure of a processing device according to the present embodiment.

FIG. 3 is a diagram showing an example of an interpretation database used in the expert panel support system according to the present embodiment.

FIG. 4 is a diagram showing details of an expert panel association table.

FIG. 5 is a diagram showing details of a case table.

FIG. 6 is a diagram showing details of an analysis table.

FIG. 7 is a diagram showing details of a cancer type table.

FIG. 8 is a diagram showing details of a gene table.

FIG. 9 is a diagram showing details of a comment table.

FIG. 10 is a diagram showing an example of an annotation table.

FIG. 11 is a diagram showing details of a variant table.

FIG. 12 is a flowchart showing an overall procedure of an expert panel support method according to the present embodiment.

FIG. 13 is a flowchart showing a procedure executed by the processing device when an expert panel support screen is created.

FIG. 14 is a flowchart showing a procedure executed by the processing device when a previous pathogenic interpretation is displayed.

FIG. 15 is a flowchart showing a detailed procedure for correcting a gene, a transcription product, and a genome position.

FIG. 16 is a flowchart showing a detailed procedure for performing sorting according to a variant position, a cancer type, and an expert panel holding date and time.

FIG. 17 is a diagram showing an example of an expert panel support screen.

FIG. 18 is a diagram showing an example of an identical variant case display portion (part 1).

FIG. 19 is a diagram showing an example of the identical variant case display portion (part 2).

FIG. 20 is a diagram showing an example of the identical variant case display portion (part 3).

FIG. 21 is a diagram showing an example of an expert panel support screen according to a second embodiment.

FIG. 22 is a diagram showing an example of a protein information display portion.

### DESCRIPTION OF EMBODIMENTS

Next, modes for implementing the invention (referred to as "embodiments") will be described in detail with reference to the drawings as appropriate.

In the present embodiment, a test refers to a gene panel test. A member of an expert panel may be referred to as a "member" as appropriate. In the present embodiment, a gene refers to a gene in which a gene variant occurs. Further, an expert panel preliminary study, which is a study performed in advance by members of an expert panel before the expert panel is held, is appropriately referred to as a "preliminary study". As described above, the expert panel is a meeting for performing medical interpretation based on a result of a cancer gene panel test and studying an optimal treatment policy.

### System Configuration

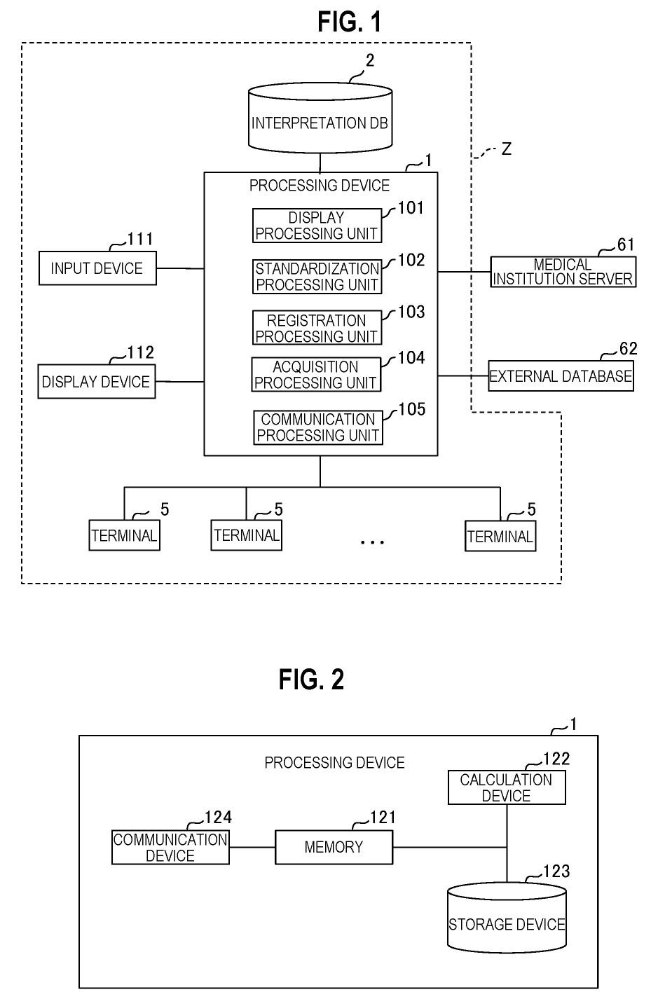

FIG. 1 is a diagram showing a configuration of an expert panel support system Z according to the present embodiment.

The expert panel support system Z includes a processing device 1, an interpretation database (DB) 2, and terminals 5. A member is a member of an expert panel. The terminal 5 is used by a member of an expert panel. A member who uses the terminal 5 may be referred to as a "user".

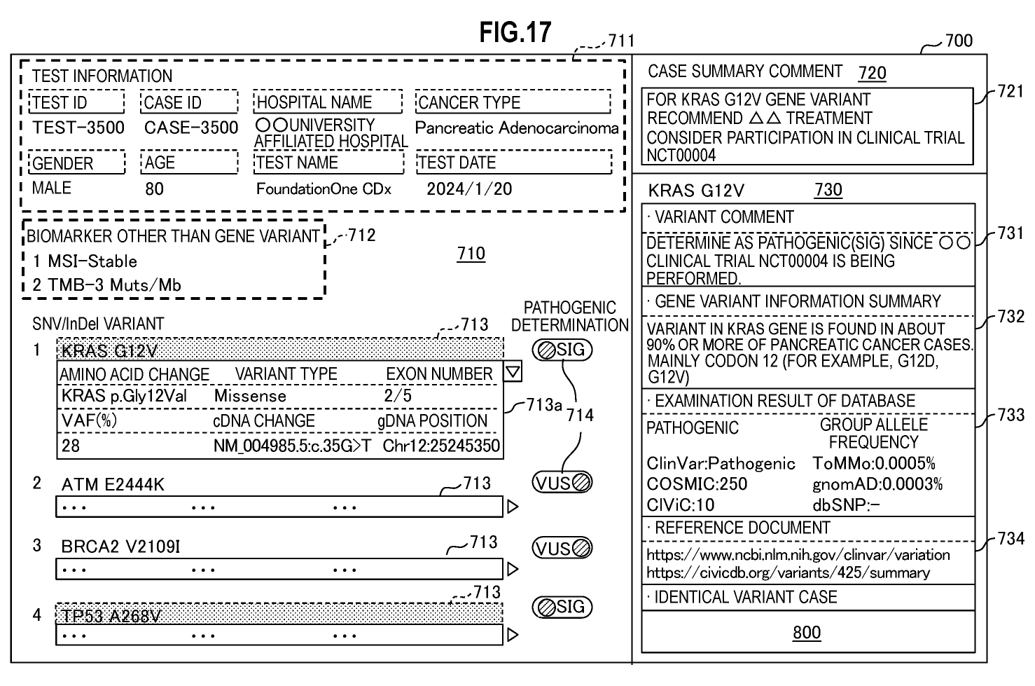

The processing device 1 creates an expert panel support screen 700 shown in FIG. 17 and causes the terminal 5 to display the created expert panel support screen 700.

The processing device 1 includes a display processing unit 101, a standardization processing unit 102, a registration processing unit 103, an acquisition processing unit 104, a communication processing unit 105, and the like.

Based on interpretation result data, the display processing unit 101 displays, on the terminal 5 used by a member who is a participant of an expert panel, a list of results of a test performed on patients (first patients) subjected to a current test for each gene variant. The standardization processing unit 102 converts gene variants that are substantially identical gene variants but are expressed by different names and variant positions between tests into information related to a representative variant. The registration processing unit 103 stores data in the interpretation database 2. The acquisition processing unit 104 that is an acquisition unit acquires interpretation result data that is data of an interpretation result for each gene variant of a patient (first patient) subjected to a gene panel test. The interpretation result is, for example, whether a variant is pathogenic and whether the variant is pathogenic cannot be distinguished. In addition, the acquisition processing unit 104 acquires information from a medical institution server 61 or the like.

The communication processing unit 105 communicates with the medical institution server 61, an external database 62, and the like.

The interpretation database 2 stores a preliminary study for a patient (first patient) subjected to a current test or a patient (second patient) previously subjected to a test before the gene panel test performed on the patient (first patient) subjected to the current test, a study result of an expert panel, and the like. The study result relates to interpretation of a gene variant, such as whether the variant is pathogenic or whether the variant is pathogenic cannot be distinguished. The interpretation database 2 may be provided in a facility where the processing device 1 is installed, or may be provided in a facility different from the facility where the processing device 1 is installed.

The processing device 1 is provided with an input device 111 such as a keyboard or a mouse, and a display device 112 such as a display.

The medical institution server 61 is a computer installed in a medical institution that performs a gene panel test, a hospital to which a doctor in charge of a patient belongs, or the like, and transmits information such as a test result of a gene panel test to the processing device 1. The medical institution server 61 often sends a test report created by a test company to the processing device 1.

The external database 62 is a database installed outside the expert panel support system Z, and is used when the processing device 1 performs various searches. Examples of the external database 62 include MANE Plus Clinical, HGNC, and HGVS.

### Hardware structure

FIG. 2 is a diagram showing a hardware structure of the processing device 1 according to the present embodiment.

The processing device 1 is implemented by a personal computer (PC) or the like, and includes a memory 121, a calculation device 122, a storage device 123, and a communication device 124.

The memory 121 is implemented by a random access memory (RAM) or the like. The calculation device 122 is implemented by a central processing unit (CPU), a graphic processing unit (GPU), and the like. The storage device 123 is implemented by a hard disk drive (HDD), a solid state drive (SSD), and the like. The communication device 124 enables communication between the terminals 5 and the medical institution server 61, the interpretation database 2, and the like.

A program stored in the storage device 123 is loaded into the memory 121. Then, the loaded program is executed by the calculation device 122 to function as units including the display processing unit 101 to the acquisition processing unit 104 shown in FIG. 1.

### Database

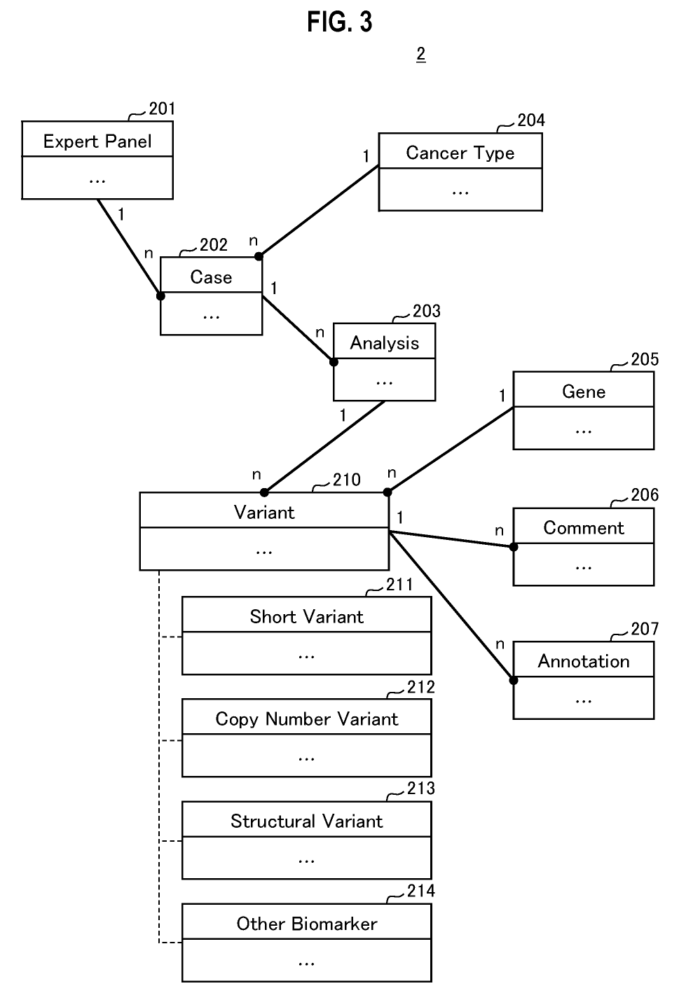

FIG. 3 is a diagram showing an example of the interpretation database 2 used in the expert panel support system Z according to the present embodiment.

The interpretation database 2 stores information related to interpretation of a gene variant, which is a study result of an expert panel. Hereinafter, interpretation of a gene variant is simply referred to as "interpretation" as appropriate. As shown in FIG. 3, the interpretation database 2 includes an expert panel association table 201 (expert panel) and a case table 202 (case). The interpretation database 2 includes an analysis table 203 (analysis), a cancer type table 204 (cancer type), and a variant table 210 (variant). The interpretation database 2 further includes a gene table 205 (gene), a comment table 206 (Comment), and an annotation table 207 (annotation). As shown in FIG. 3, tables including the expert panel association table 201 to the annotation table 207 and the variant table 210 are associated in a relational database format. However, the expert panel association table 201 to the annotation table 207 and the variant table 210 may not be associated in the relational database format.

As shown in FIG. 3, the variant table 210 includes a short variant table 211 (short variant) and a copy number variant table 212 (copy number variant) as child tables. The variant table 210 further includes a structural variant table 213 (structural variant) and an other biomarker table 214 (other biomarker). In the present embodiment, it is assumed that the variant table 210 and tables including the short variant table 211 to the other biomarker table 214 have an inheritance relationship, but are not limited to the inheritance relationship.

In FIG. 3, "1" and "n" indicate that one piece of data is associated with a plurality of pieces of data. For example, in a line connecting the expert panel association table 201 and the case table 202, "1" is shown on the expert panel association table 201 side, and "n" is shown on the case table 202 side. This indicates that one piece of data of the expert panel association table 201 is associated with a plurality of pieces of data of the case tables 202. The same applies to other tables.

### Expert Panel Association Table 201

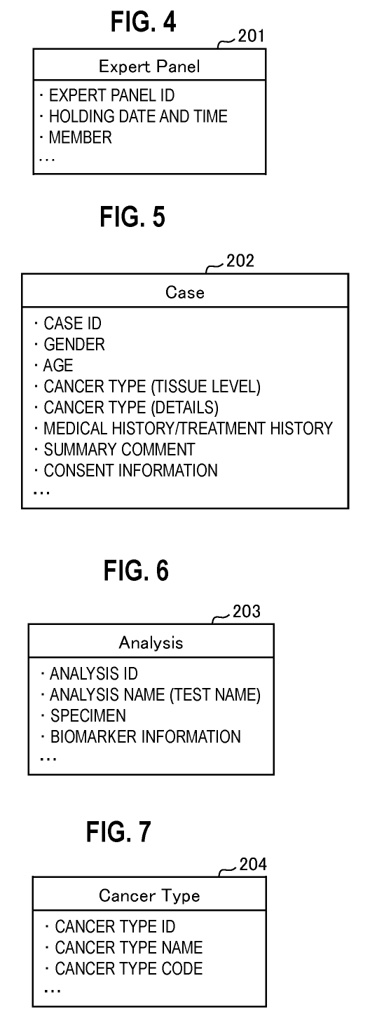

FIG. 4 is a diagram showing details of the expert panel association table 201.

The expert panel association table 201 stores information related to an expert panel, such as an expert panel ID, an expert panel holding date and time, and members.

### Case Table 202

FIG. 5 is a diagram showing details of the case table 202.

The case table 202 stores information such as a case ID, gender, age, a cancer type (tissue level), and a cancer type (details) of a patient subjected to a test (gene panel test). Information such as medical history/treatment history, summary comment, and consent information of a patient subjected to a test is stored. The summary comment is a comment input by a doctor in charge for a case. The doctor in charge is a doctor in charge when the test is performed, and is not a member of an expert panel in many cases. The cancer type (tissue level) and the cancer type (details) are input by a doctor in charge (not a member of an expert panel) based on a result of the test.

### Analysis Table 203

FIG. 6 is a diagram showing details of the analysis table 203.

The analysis table 203 stores information on an analysis result of a test performed on a patient. Information described in a report created by a test company (not shown) is stored as the analysis result.

The analysis table 203 stores an analysis ID, an analysis name (test name), information on a specimen and the like, biomarker information, and the like. The specimen is a specimen collected from a patient. The biomarker information is information stored when a biomarker other than a short variant, a copy number variant, and a structural variant is detected as a result of the test.

### Cancer Type Table 204

FIG. 7 is a diagram showing details of the cancer type table 204.

The cancer type table 204 stores data related to a type of a cancer (cancer type).

The cancer type table 204 stores a cancer type ID, a cancer type name, a cancer type code, and the like.

### Gene Table 205

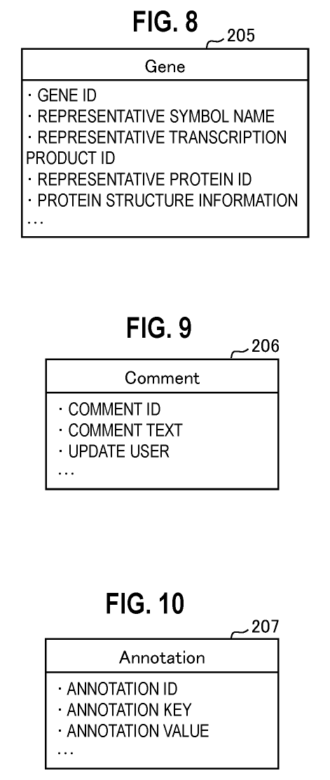

FIG. 8 is a diagram showing details of the gene table 205.

The gene table 205 stores information on a gene, a transcription product generated by the stored gene, and information on a protein.

The gene table 205 stores a gene ID, a representative symbol name, a representative transcription product ID, a representative protein ID, protein structure information, and the like. The symbol name is named for a gene. For example, there is "KRAS" which is one of cancer genes. The representative symbol name is a symbol name having a highest priority.

The transcription product is RNA generated from DNA corresponding to a gene. The representative transcription product ID is a used ID having a highest priority among transcription product IDs of the gene. Similarly, the representative protein ID is a used protein ID having a highest priority in the gene.

The gene table 205 stores a representative amino acid change name and a representative cDNA change name in addition to the above information.

### Comment Table 206

FIG. 9 is a diagram showing details of the comment table 206.

The comment table 206 stores data related to a comment of a member of an expert panel.

The comment table 206 stores information such as a comment ID, a comment text which is a comment, and an update user which is information on a person who updated the comment.

### Annotation Table 207

FIG. 10 is a diagram showing an example of the annotation table 207.

The annotation table 207 is an annotation for a gene variant.

The annotation table 207 stores an annotation ID, an annotation key, an annotation value, and the like. The annotation value is a main body of annotation information, and stores an analysis result or external database information associated with a gene variant found by a biological information analysis or examination. The annotation key stores a key for classifying an annotation value. The annotation key includes "ClinVar" and "COSMIC" in the case of external database information.

The representative symbol name, the representative transcription product ID, and the representative protein ID are standardized as will be described later.

Although there are international representative names for gene variant names and the like, names different from the representative names may be used for the following reasons and the like.

### (1) Difference in Reference Genomes

A position of a reference genome may be updated when the genome is re-read. For example, "GRCh37 12: 25398234" corresponds to "GRCh38 12: 25245350". "GRCh37" and "GRCh38" indicate versions of international human genome reference sequences. "12" indicates a chromosome number, and "25398234" and "25245350" indicate positions on the chromosome.

That is, positions of substantially identical gene variants are described as "25398234" or "25245350" depending on a version. In a test result, gene variant positions may be described in an old version. However, it may be necessary to use a latest version in an expert meeting according to the present embodiment. In such a case, a position of a gene variant described as "GRCh37 12: 25398234" needs to be converted into "GRCh38 12: 25245350".

### (2) Difference in Gene Names

For example, "CD274" and "PD-L1" indicate an identical gene. "CD274" indicates a gene name, and "PD-L1" indicates a protein expressed by "CD274". A gene name is used by a medical institution, or a protein expressed by a gene is commonly used as an alias of the gene name. Therefore, when "PD-L1" is used as a test result, it is necessary to convert "PD-L1" into "CD274" to unify gene information.

The same applies to "ERBB2" and "HER2". In this case, "ERBB2" is a gene name, and "HER2" indicates a protein expressed by "ERBB2". In this case, it is necessary to convert "HER2" into "ERBB2" as a test result.

### (3) Difference in Transcript (Transcription Product)

A transcript (transcription product) to be used may differ depending on a medical institution. "NP_001341538.1: p. Val600G lu" and "NP_001361173.1: p. Val640G lu" indicate a range of proteins derived from identical gene variants. A difference between these two notations is a difference depending on which protein derived from which transcript is used as a reference. The standardization processing unit 102 unifies such differences in transcripts.

### (4) Difference in Simple Notations

Even identical gene variants may have different notations depending on a medical institution. For example, a certain gene variant may be described as "p. Gly12A sp" in a certain medical institution, but may be abbreviated as "p. G12D" in another medical institution. Thus, when a notation of a gene variant differs depending on a medical institution, the notation needs to be unified.

As described above, even information on substantially identical gene variants may have different notations such as gene names and variant positions depending on a test. Therefore, in the present embodiment, different names depending on a medical institution are converted into representative information (representative symbol name, representative transcription product ID, and the like) by standardization processing to be described later.

An alias in the annotation table 207 refers to a name other than a representative name.

### Variant Table 210

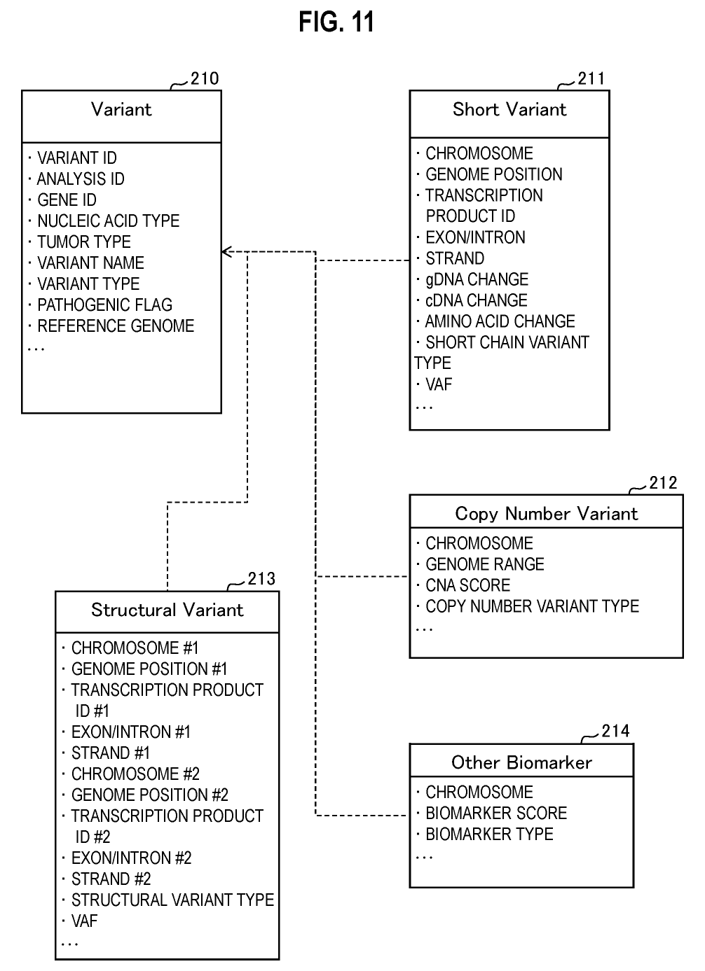

FIG. 11 is a diagram showing details of the variant table 210.

As described above, the variant table 210 includes the short variant table 211, the copy number variant table 212, the structural variant table 213, the other biomarker table 214, and the like as child tables.

The variant table 210 stores a variant ID that is identification information for a gene variant, an analysis ID that is identification information for an analysis result, and a gene ID that is identification information for a gene. The variant table 210 further stores a nucleic acid type, a tumor type, a variant name, a variant type, a pathogenic flag, a reference genome, and the like. The variant is a gene variant. The pathogenic flag is a flag for determining pathogenic significance by a member of an expert panel. The pathogenic significance includes determining whether a variant is pathogenic, whether a variant is pathogenic cannot be distinguished, and the like.

The information stored in the variant table 210 is information described in a test result.

Hereinafter, a gene variant to be treated is simply referred to as a gene variant.

### Short Variant Table 211

The short variant table 211 stores a chromosome (chromosome number) at which a gene variant is located, a genome position of the gene variant, and a transcription product ID which is identification information of a transcription product generated by the gene variant. The short variant table 211 stores an exon/intron, a strand, a gDNA change, a cDNA change, an amino acid change caused by a gene variant, and the like in a gene variant. In addition, a short chain variant type in a gene variant, a variant allele frequency (VAF), and the like are stored. Information on a structure of an exon or an intron is stored in the item of exon/intron.

### Copy Number Variant Table 212

The copy number variant table 212 stores a chromosome (chromosome number) at which a gene variant of a copy number variant occurs, a genome range in which a copy number variant occurs, a CNA score, and a copy number variant type. CNA is a copy number change serving as a somatic cell variant in a tumor, and the CNA score indicates a level of the occurrence of CNA.

### Structural Variant Table 213

The structural variant table 213 stores a chromosome at which a structural variant occurs, a genome position, a transcription product ID which is identification information of a transcription product generated by the structural variant, and the like. In addition, information on an exon/intron, a strand, and the like in a structural variant is stored. When a structural variant is fusion of two genes, information on the two genes to be fused is required. In such a case, as shown in FIG. 11, a plurality of pieces of information may be stored for one structural variant, such as "chromosome #1" to "strand #1" and "chromosome #2" to "strand #2". Although the "chromosome" to the "strand" are each stored with the number of two in the structural variant table 213 in the example shown in FIG. 11, the "chromosome" to the "strand" may each be stored with the number of one in the structural variant table 213, or may each be stored with the number of three in the structural variant table 213. A plurality of pieces of information may be stored for any one of the "chromosome" to the "strand" in the structural variant table 213.

Information on a structure of an exon or an intron is stored in the exon/intron (exon/intron #1, exon/intron #2).

The structural variant table 213 stores information such as a structural variant type and a variant allele frequency (VAF).

### Other Biomarker Table 214

The biomarker table stores information on a biomarker other than a short variant, a copy number variant, and a structural variant.

Specifically, information such as a gene in which a target biomarker is present, a biomarker score indicating a level of occurrence of the biomarker, and a biomarker type is stored.

### Flowchart

### Overall Processing

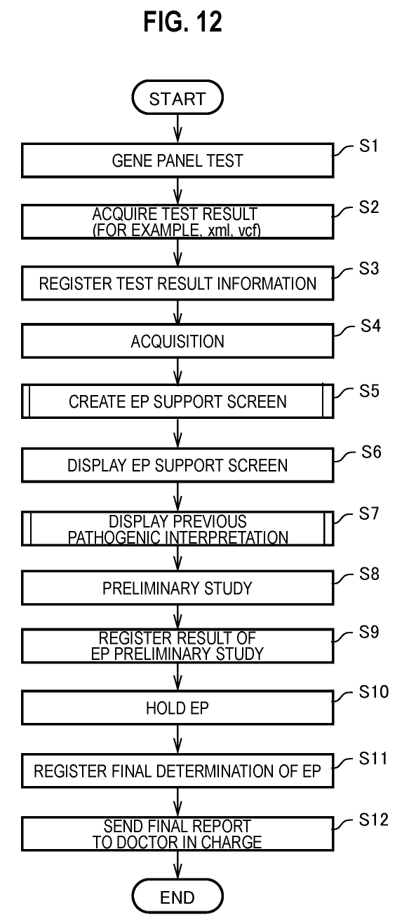

FIG. 12 is a flowchart showing an overall procedure of an expert panel support method according to the present embodiment.

First, a test (gene panel test) is performed on a patient (S1). The test is generally performed on one patient a plurality of times.

Then, the communication processing unit 105 acquires test result information of the test from the medical institution server 61 (S2). As described above, since the test is generally performed a plurality of times, the test result information acquired in step S2 is information on test results of the test performed a plurality of times. The test result information acquired in step S2 is test result information related to one patient. The test result information is generally in a file format such as an xml file or a vcf file, but may be in other file formats.

Subsequently, the registration processing unit 103 stores the test result information acquired in step S2 in the case table 202 shown in FIG. 5, the analysis table 203 shown in FIG. 6, and the like (S3).

Subsequently, when a preliminary study is performed, the acquisition processing unit 104 acquires, from the interpretation database 2, interpretation result data that is data of an interpretation result for each gene variant of a patient (first patient) subjected to a gene panel test (S4). Step S4 is an "acquisition step".

Then, the display processing unit 101 creates the expert panel support screen (EP support screen) 700 (see FIG. 17) (S5). Details of step S5 will be described later.

Then, the display processing unit 101 displays the created expert panel support screen (EP support screen) 700 on the terminal 5 used by a member who performs the preliminary study (S6). Step S6 is a display processing step of displaying a list of results of the test performed on the first patient for each gene variant on the terminal 5 used by a participant of an expert panel based on the interpretation result data.

The list is simply described such that all contents can be understood. In the present embodiment, as shown in FIG. 17, a name of a gene variant detected as a result of an analysis in the test is displayed in a variant name display portion 713. In the list, information of a gene variant displayed in the variant name display portion 713 is displayed in variant summary information 713a shown in FIG. 17. Further, in the list, a summary comment for a gene variant is displayed in a summary comment display portion 720 shown in FIG. 17.

The preliminary study is performed in advance by each member of the expert panel before the expert panel is held. In the preliminary study, each member considers a recommended treatment proposal. At this time, each member considers a recommended treatment proposal based on a result of a previous test, a result of an expert panel held in the past, a result of a current test, and the like.

A variant level is also displayed on the expert panel support screen 700. The variant level indicates an abnormality level of a variant. Examples of the variant level include an allele frequency, a copy number of a gene in a gene copy variant, and a read number (expression level). Examples of a variant include a gene variant and an amino acid change.

The display processing unit 101 displays previous pathogenic interpretation for a gene variant on the expert panel support screen 700 (S7). The pathogenic interpretation includes whether a variant is pathogenic. Step S7 is a "display processing step".

Based on the expert panel support screen 700 displayed in step S7, each member performs a preliminary study (S8).

Then, each member who performed the preliminary study registers a result of the preliminary study in the interpretation database 2 via the terminal 5 used by the member (S9). The registered information on the result of the preliminary study is registered in the comment table 206 shown in FIG. 9 or the variant table 210 shown in FIG. 11 by the registration processing unit 103.

Then, the expert panel (EP) is performed (S10). In the expert panel, members of the expert panel discuss a treatment policy, a recommended medicine, the necessity of clinical trial, and the like based on results of the preliminary study and the like. The expert panel support screen 700 is also displayed on the terminal 5 or the like in the expert panel. The processing in steps S5 and S6 is performed before step S10.

When the expert panel ends, the members of the expert panel registers a final determination of the expert panel (EP) (S11). The registration is performed via the terminals 5 used by the members of the expert panel. Then, the registered information is stored in the interpretation database 2 shown in FIG. 12. The registered information is pathogenic interpretation or the like. The information on the final determination is stored in the interpretation database 2 by the registration processing unit 103.

The communication processing unit 105 creates a final report based on the information registered in step S11, and sends the created final report to a doctor in charge (S12). Specifically, the acquisition processing unit 104 of the processing device 1 transmits the information on the final report to the medical institution server 61 installed in a medical institution to which the doctor in charge belongs.

### Creation of Expert Panel Support Screen 700

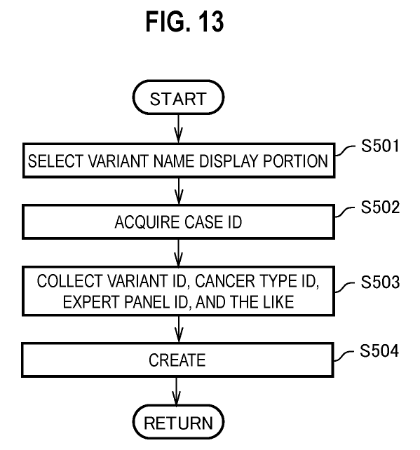

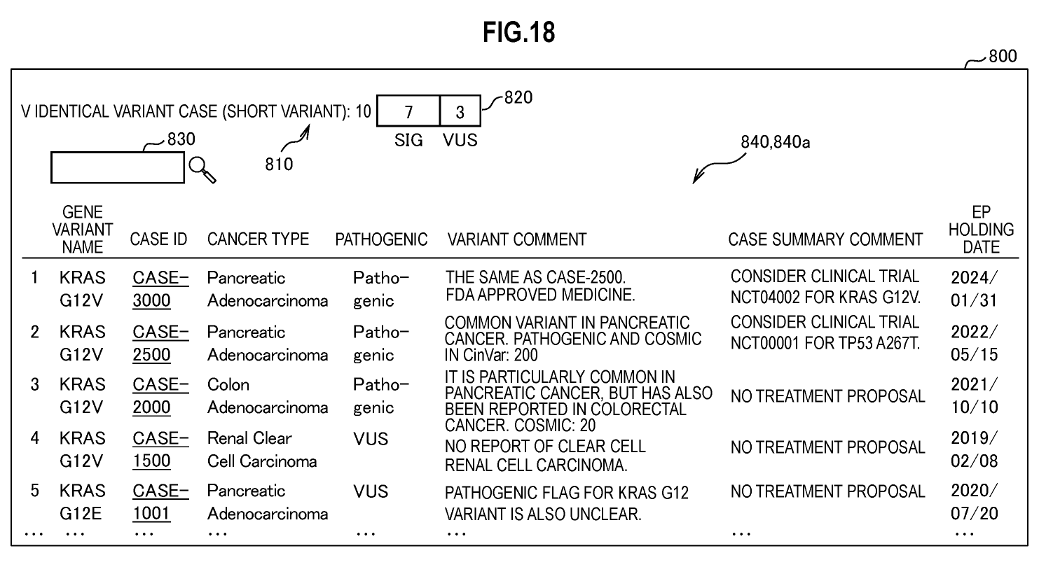

FIG. 13 is a flowchart showing a procedure executed by the processing device 1 when the expert panel support screen 700 is created. FIG. 13 is a diagram showing details of the processing in step S5 in FIG. 12. In FIG. 13, in particular, a creation procedure of an identical variant case display portion 800 shown in FIGS. 18 to 20 will be described.

First, a user selects the variant name display portion 713 shown in FIG. 17 via the input device 111 (S501).

Subsequently, the display processing unit 101 extracts all case IDs having the same variant ID as the selected variant name display portion 713 from the case table 202 (S502). There may be only one case ID or a plurality of case IDs extracted in step S502.

Then, the display processing unit 101 collects a variant ID, a cancer type ID, an expert ID, and the like of another patient associated with the case ID extracted in step S502 in (S503). The variant ID is collected from the variant table 210, the cancer type ID is collected from the cancer type table, and the expert ID is collected from the expert panel association table 201. Information associated with the variant ID includes a "pathogenic flag". The pathogenic flag is an "interpretation result of gene variants of a plurality of second patients".

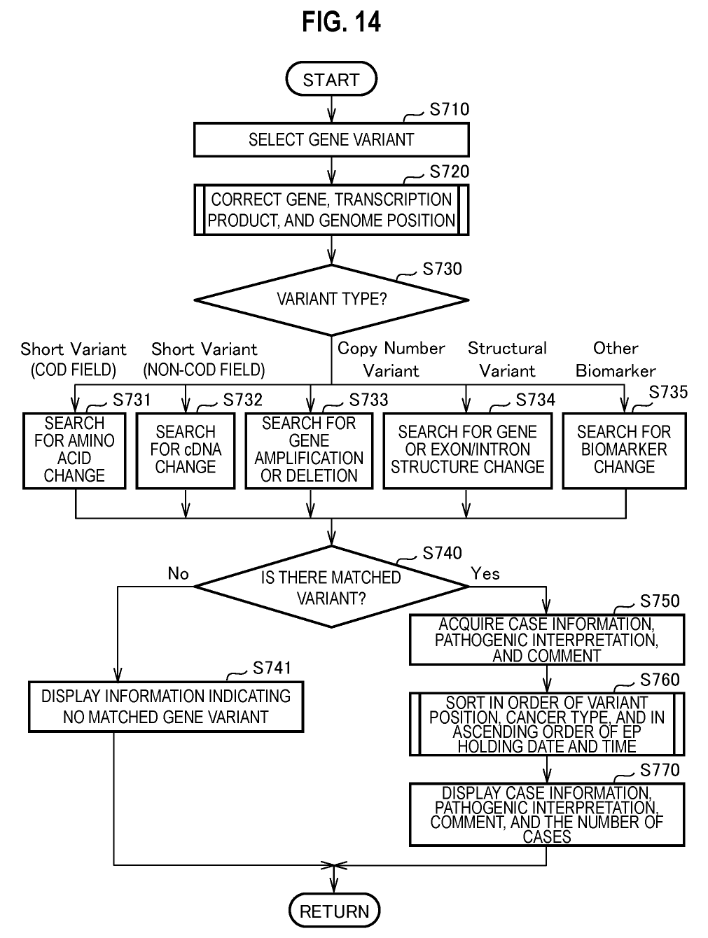

In step S6 of FIG. 12, the display processing unit 101 creates an identical variant information display portion 840 of the identical variant case display portion 800 shown in FIGS. 18 to 20 based on the information collected in steps S502 and S503 (S504). In step S6 of FIG. 12, the information collected in steps S502 and S503 is information associated with the case ID, the variant ID, the cancer type ID, and the expert ID collected in steps S502 and S503 in the interpretation database 2. In step S504, a screen on which the collected information is displayed is created, and contents displayed in processing of FIG. 14 to be described later is sorted.

Steps S502 and S503 are "acquisition steps".

### Display of Previous Pathogenic Interpretation

FIG. 14 is a flowchart showing a procedure executed by the processing device 1 when past pathogenic interpretation is displayed. FIG. 14 is a diagram showing details of the processing of step S7 in FIG. 12.

First, the display processing unit 101 selects a gene variant to be processed (S710).

Subsequently, the standardization processing unit 102 corrects a gene, a transcription product, and a genome position (S720). Although processing of step S720 will be described later, a symbol name of a gene in which a gene variant occurred and information on a transcription product are standardized.

Subsequently, the display processing unit 101 determines a variant type (S730). In step S730, the display processing unit 101 makes a determination of step S730 with reference to the item of variant type in the variant table 210 in FIG. 11. The variant type is information described in a test result which is registered in the variant table 210.

In the following description, "match" refers to that information of a gene variant matches information of the gene variant selected in step S710 of FIG. 14.

When a variant type is a short variant code region or the like (S730 → short variant (code region)), the display processing unit 101 searches for an amino acid change (S731). In step S731, the display processing unit 101 refers to the short variant table 211 in FIG. 11 and acquires a record in which an amino acid position of an amino acid change (HGVSp) matches the selected gene variant by using a standardized representative symbol name and a representative transcription product ID as keys.

When a variant type is a short variant non-code region or the like (S730 → short variant (non-code region), the display processing unit 101 searches for a cDNA change (S732). In step S732, the display processing unit 101 refers to the short variant table 211 in FIG. 11, and acquires a record in which a cDNA position in the item of cDNA change (HGVSc) matches the selected gene variant by using a standardized representative symbol name and a representative transcription product ID as keys.

When a variant type is a copy number variant (S730 → copy number variant), the display processing unit 101 searches for gene amplification or deletion (S733). In step S733, the display processing unit 101 refers to the copy number variant table 212 in FIG. 11 and acquires a record in which a change (amplification or deletion) matches the selected gene variant by using a standardized representative symbol name as a key. The change (amplification or deletion) is specifically a CNA score.

When a variant type is a structural variant (S730 → structural variant), the display processing unit 101 searches for a gene or an exon/intron structural change (S734).

Processing of step S734 varies depending on a structural variant type. The structural variant type is determined by an item of the structural variant type in the structural variant table 213 shown in FIG. 11.

When the structural variant type is fusion, the display processing unit 101 acquires a record in which both a representative symbol name standardized for an upstream gene and a representative symbol name standardized for a downstream gene match between the structural variant type (fusion) and the selected variant. "Matching" refers to matching with the gene variant selected in step S710. Depending on a gene variant, the display processing unit 101 also returns a record in which only a cancer gene or a cancer suppression gene matches with the selected gene variant.

When the structural variant type is deletion, insertion, duplication, inversion, or complicated rearrangement, the display processing unit 101 acquires a record in which a structural variant type and exon/intron match the selected gene variant by using a standardized representative symbol name and a representative transcription product ID as keys. The record is a record of the structural variant table 213.

When the variant type is other biomarker (other biomarker) (S730 → other biomarker), the display processing unit 101 searches for a biomarker change (S735). In step S735, the display processing unit 101 acquires a record in which a biomarker change (labels such as a high value, a median value, and a low value) matches the selected gene variant by using a standardized representative symbol name as a key. The record is a record of the other biomarker table 214. Specific examples of the biomarker change include an MSI score, a TMB score, and an LOH score.

By executing the processing in steps S731 to S735, appropriate matching can be performed for each gene variant type.

Subsequently, the display processing unit 101 determines whether there is a matched gene variant (variant) as a result of the processing in steps S731 to S735 (S740).

When there is no matched gene variant (No in S740), the display processing unit 101 displays that there is no matched gene variant in the variant table 210 and a child table of the variant table 210 (S741). In step S741, the display processing unit 101 may display a message for prompting a user to refer to a document or the like.

When there is a matched gene variant (Yes in S740), the display processing unit 101 acquires case information, pathogenic interpretation information, and comment information related to the matched gene variant from the interpretation database 2 (S750). The case information is information stored in the case table 202 in FIG. 5, and the pathogenic interpretation information is a pathogenic flag stored in the variant table 210 in FIG. 11. The comment information is information stored in the comment table 206 of FIG. 9.

Subsequently, the display processing unit 101 sorts the information acquired in step S750 in order of a variant position, a cancer type, and in ascending order of an expert panel (EP) holding date and time (S760). The processing of step S760 will be described later.

Then, the display processing unit 101 displays the case information, the pathogenic interpretation information, the comment information, and the number on the terminal 5 in the order sorted in step S760 (S770). The number is the number of matched gene variants in steps S731 to S735.

Correction of Gene, Transcription Product, and Genome Position

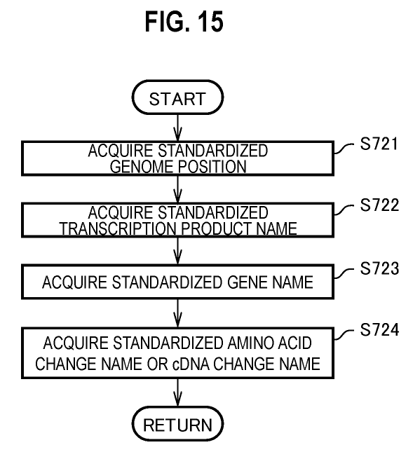

FIG. 15 is a flowchart showing a detailed procedure of step S720 (correction of a gene, a transcription product, and a genome position) in FIG. 14.

As described above, there are variations in notation such as a genome version and a gene name for each test. Therefore, in the correction of a gene, a transcription product, and a genome position, these notation variations are standardized and used for later searches.

First, the standardization processing unit 102 acquires a standardized genome position for a genome position of a gene variant to be processed (S721). The standardization processing unit 102 acquires a standardized transcription product name by searching the external database 62 or the like. A version of a genome position may be different for each test, such as GRCh37 or GRCh38. In step S721, the standardization processing unit 102 unifies a version of a genome position to any version (in many cases, the latest version). The processing of step S721 may be executed by the standardization processing unit 102 with reference to the annotation table 207.

Subsequently, the standardization processing unit 102 acquires a standardized transcription product name for a transcription product of the gene variant to be processed (S722). The standardization processing unit 102 acquires the standardized transcription product name by searching the external database 62 (for example, MANE Plus Clinical) or the like. Matched Annotation from NCBI and EMBL-EBI (MANE) is a list defining most representative clinical transcription product names of Refseq and Ensembl/GENCODE among transcription products of genes. The standardization processing unit 102 specifies a representative transcription product ID based on the acquired transcription product name.

Then, the standardization processing unit 102 acquires a standardized gene name for the transcription product of the gene variant to be processed (S723). The standardization processing unit 102 acquires a most standard gene name (symbol name) by searching the external database 62 (for example, HGNC) or the like. The standardization processing unit 102 sets the acquired gene name as a representative symbol name.

Further, the standardization processing unit 102 acquires a standardized amino acid change name or cDNA change name for the transcription product of the gene variant to be processed (S724). The standardization processing unit 102 searches the external database 62 (for example, HGVS or the like) or the like to unify variants in description methods using an amino acid change name or a cDNA change name into a predetermined representative amino acid change name or representative cDNA change name.

Results output by the processing in steps S721 to S724 are stored in the gene table 205 shown in FIG. 5. By the processing in steps S721 to S724, gene variants that are substantially identical but are expressed by different names and variant positions between tests are displayed as identical variants on the expert panel support screen 700 shown in FIG. 17. Accordingly, it is possible to unify various expressions, and it is possible to improve study efficiency.

Sorting according to Variant Position, Cancer Type, And Expert Panel Holding Date And Time

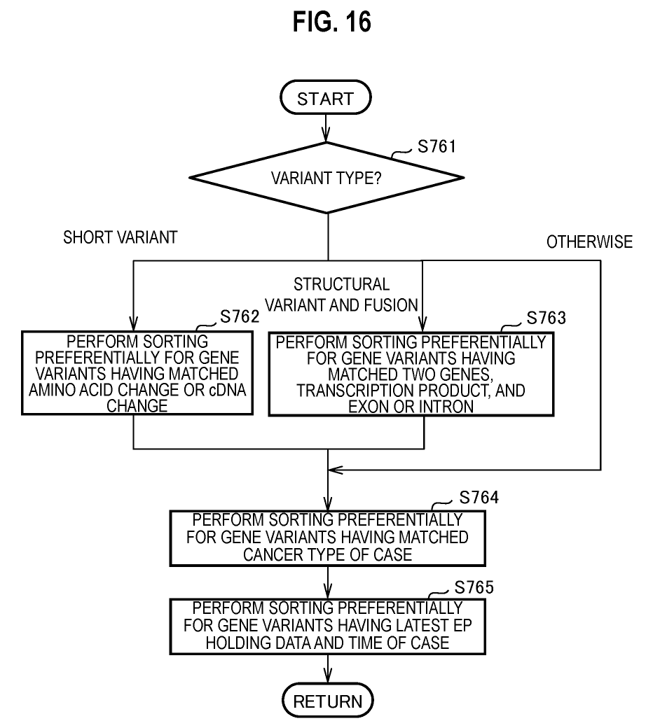

FIG. 16 is a flowchart showing a detailed procedure in step S760 (sorting according to a variant position, a cancer type, and an expert panel holding date and time) shown in FIG. 14.

The following processing is executed for the matched gene variants in steps S731 to S735 in FIG. 14.

First, the display processing unit 101 determines a variant type of a gene variant (S761). The variant type is determined by the display processing unit 101 searching a variant type item in the variant table 210 in FIG. 11.

When the variant type is a short variant (S711 → short variant), the display processing unit 101 performs sorting preferentially for gene variants having a matched amino acid change or cDNA change (S762). Thereafter, the display processing unit 101 advances the processing to step S764.

When the variant type is a structural variant and fusion (S761 → structural variant and fusion), the display processing unit 101 performs sorting preferentially for gene variants having matched two genes, a matched transcription product, and a matched exon or intron (S763). Two genes refers to two genes to be fused. Thereafter, the display processing unit 101 advances the processing to step S764.

When the variant type is other than the above gene variants (S761 → otherwise), the display processing unit 101 advances the processing to step S764. The term "otherwise" refers to that the variant type is neither a short variant nor a fusion.

In step S764, the display processing unit 101 refers to the case table 202 and performs sorting preferentially for gene variants having a matched cancer type. When step S762 or step S763 is performed, sorting in step S764 is performed in addition to sorting in steps S762 and S763.

Then, the display processing unit 101 refers to the expert panel association table 201 and performs soring in ascending order of expert panel (EP) holding date and time (S765).

### Expert Panel Support Screen 700

FIG. 17 is a diagram showing an example of the expert panel support screen 700.

The expert panel support screen 700 is a screen displayed on the terminal 5 at the time of a preliminary study or when an expert panel is held.

The expert panel support screen 700 includes a basic information display portion 710, the summary comment display portion 720, and a variant detail display portion 730.

The basic information display portion 710 displays a test information display portion 711, a biomarker display portion 712, the variant name display portion 713, and a pathogenic determination button 714.

The test information display portion 711 displays information such as a test ID, a case ID, a name of a hospital where a test is performed, a result of the test, a suspected cancer type, gender and age of a patient, a test name, and a test date. The test information display portion 711 is displayed based on information stored in the case table 202 shown in FIG. 5 or the cancer type table 204.

The biomarker display portion 712 displays information related to a biomarker other than a gene variant detected by a test. The biomarker other than a gene variant is detected in a test, and is stored in information sent from a test company. The biomarker display portion 712 is displayed based on information extracted from other biomarker information by using the biomarker information stored in the analysis table 203 in FIG. 6 as a key.

The variant name display portion 713 displays a name (representative symbol name) of a gene variant detected as an analysis result in a test. The variant name display portion 713 displays a gene name and the like extracted from the gene table 205 shown in FIG. 8 by the display processing unit 101 based on an analysis ID shown in FIG. 6 and the variant table 210 shown in FIG. 11. As described above, contents of the analysis table 203 shown in FIG. 6 are stored based on a report created by a test company. Therefore, information displayed in the variant name display portion 713 is based on the report created by the test company.

When a user clicks a pull-down button, the variant summary information 713a is displayed. The variant summary information 713a displays data stored in the gene table 205, the variant table 210, and the short variant table 211 to the structural variant table 213, which are child tables of the variant table 210.

A user can register a determination regarding a pathogenic variant (pathogenic determination) in the pathogenic flag of the variant table 210 shown in FIG. 11 by operating the pathogenic determination button 714. In the diagram shown in FIG. 17, the pathogenic determination button 714 is implemented by a toggle button, but is not limited to the toggle button, and may be implemented by a radio button, a check box, or the like. With the pathogenic determination button 714, a user can select and input pathogenic (SIG), whether a variant is pathogenic cannot be distinguished (VUS), or the like.

Then, a gene variant determined to be pathogenic (SIG) by the pathogenic determination button 714 is highlighted in the variant name display portion 713 by adding a background color or the like. In FIG. 17, the highlight display is represented by a dot.

Then, when a user selects a predetermined gene variant by clicking or the like in the variant name display portion 713, the summary comment display portion 720 and the variant detail display portion 730 are displayed as detailed information related to the selected gene variant. In FIG. 17, the summary comment display portion 720 and the variant detail display portion 730 are displayed on a side panel of the basic information display portion 710, but are not limited thereto. For example, the summary comment display portion 720 and the variant detail display portion 730 may be displayed as separate windows different from the basic information display portion 710.

A case summary comment display portion 721 is displayed in the summary comment display portion 720. The case summary comment display portion 721 displays a summary comment for the selected gene variant, which is input by a doctor in charge. A summary comment is stored in the summary comment of the case table 202 shown in FIG. 5.

The variant detail display portion 730 includes a variant comment input portion 731, a gene variant information summary display portion 732, an examination result display portion 733, a reference document display portion 734, the identical variant case display portion 800, and the like.

A user (member) can input a comment related a gene variant to the variant comment input portion 731. The comment is a preliminary study, or a test result (interpretation result) from an expert panel. The input comment is stored in the comment table 206 shown in FIG. 9. Accordingly, a gene variant to be processed is associated with a comment. When a preliminary study is performed, a comment input by a user in the preliminary study may be displayed in the variant comment input portion 731. In this case, when an expert panel is performed, the comment is added or edited in the variant comment input portion 731.

The gene variant information summary display portion 732 displays a summary related to the gene variant selected in the variant name display portion 713. The information displayed in the gene variant information summary display portion 732 is displayed by the display processing unit 101 with reference to the external database 62. A result of creation AI or the like may be displayed on the gene variant information summary display portion 732. The external database 62 is a database installed outside the expert panel support system Z.

Then, the examination result display portion 733 displays a result of searching for information related to a target gene variant in the external database 62 of the expert panel support system Z. The search is performed by the display processing unit 101. In the example shown in FIG. 17, a pathogenic flag and a group allele frequency are displayed. The examination result display portion 733 may display a manual examination result.

The reference document display portion 734 displays information extracted by searching the external database 62 of the expert panel support system Z by the display processing unit 101. The reference document display portion 734 displays a site provided by the external database 62 or a link to a genome browser.

Then, the identical variant case display portion 800 displays contents studied in a previous expert panel for a gene variant which is identical with the gene variant selected in the variant name display portion 713. The identical variant case display portion 800 will be described later with reference to FIGS. 18 to 20.

When an expert panel is held, the new expert panel support screen 700 different from that at the time of a preliminary study may be displayed. In this case, the expert panel support screen 700 in which a result of the preliminary study is input may be displayed on the terminal 5 separately from the expert panel support screen 700 for the expert panel.

### Identical Variant Case Display Portion 800

FIGS. 18 to 20 are diagrams showing examples of the identical variant case display portion 800.

As shown in FIGS. 18 to 20, the identical variant case display portion 800 includes an identical variant case number display portion 810, a pathogenic determination ratio display portion 820, a search portion 830, and the identical variant information display portion 840. Information necessary for the display of the identical variant case display portion 800 is acquired by the processing shown in FIG. 13.

A screen of the identical variant case display portion 800 shown in FIGS. 18 to 20 may be displayed in the identical variant case display portion 800 shown in FIG. 17. Alternatively, when a user clicks the identical variant case display portion 800 in FIG. 17, the screen of the identical variant case display portion 800 shown in FIGS. 18 to 20 may be pop-up displayed.

In this manner, the identical variant case display portion 800 is displayed on the expert panel support screen 700, or is pop-up displayed. Accordingly, interpretation results of a gene variant of a plurality of patients (second patients) previously subjected to a test and interpretation results of a gene variant of a patient (first patient) subjected to a current test are displayed in association with each other. An interpretation result indicates whether a gene variant is pathogenic (SIG), or whether a gene variant is pathogenic cannot distinguished (VUS).

The interpretation result of the gene variant of the first patient (patient subjected to a current test) and the interpretation result of the gene variant of the second patients (patients subjected to a test in the past) are displayed in association with each other in the identical variant case number display portion 810. In this case, based on interpretation result data, information related to the number of gene variants specified for the second patients is displayed in the identical variant case number display portion 810. Specifically, the identical variant case number display portion 810 displays the number of cases studied in a previous expert panel for a gene variant which is identical with the gene variant selected in the variant name display portion 713 in FIG. 17. That is, the identical variant case number display portion 810 displays the number of target gene variants stored in the interpretation database 2. The gene variant selected in the variant name display portion 713 in FIG. 17 is appropriately referred to as a "selected gene variant". By displaying the identical variant case number display portion 810, a user can easily recognize how many identical gene variants have been detected in the past.

The pathogenic determination ratio display portion 820 displays the number of (SIG) in which a gene variant is determined to be pathogenic and the number of (VUS) in which whether a gene variant is determined to be pathogenic cannot be distinguished for the number of cases displayed in the identical variant case number display portion 810. The number displayed in the pathogenic determination ratio display portion 820 is the number of cases studied in a previous expert panel. As described above, the pathogenic determination ratio display portion 820 displays the number of cases in which a gene variant is determined to be pathogenic and the number of cases in which an otherwise determination is made (whether a gene variant is pathogenic cannot be distinguished) in a distinguished manner for the interpretation results for the patients (second patient) subjected to a previous test. With the pathogenic determination ratio display portion 820, a user can easily confirm which gene variant is determined to be pathogenic among gene variants detected in the past. Accordingly, the user can refer to pathogenic determination for a gene variant detected in a current test.

When a user inputs a search word to the search portion 830, information that matches the input search word is displayed in the identical variant information display portion 840.

The identical variant information display portion 840 displays a study result in a previous expert panel on a gene variant that is identical with the selected gene variant.

Information displayed in the identical variant information display portion 840 differs depending on a type of a gene variant.

For example, when the gene variant selected in the variant name display portion 713 in FIG. 17 is a short variant, a gene variant name and a case ID are displayed in an identical variant information display portion 840a as shown in the identical variant information display portion 840a in FIG. 18. The identical variant information display portion 840a displays a cancer type, a pathogenic flag, a variant comment, a case summary comment, an expert panel (EP) holding date, and the like.

When the gene variant is a short variant, an amino acid change and a cDNA change that match the gene variant selected in the variant name display portion 713 in FIG. 17 are displayed in the identical variant information display portion 840a. When there is no gene variant whose amino acid change or cDNA change matches the gene variant selected in the variant name display portion 713 in FIG. 17, a gene variant whose amino acid position or cDNA position matches the selected gene variant is displayed as a reference.

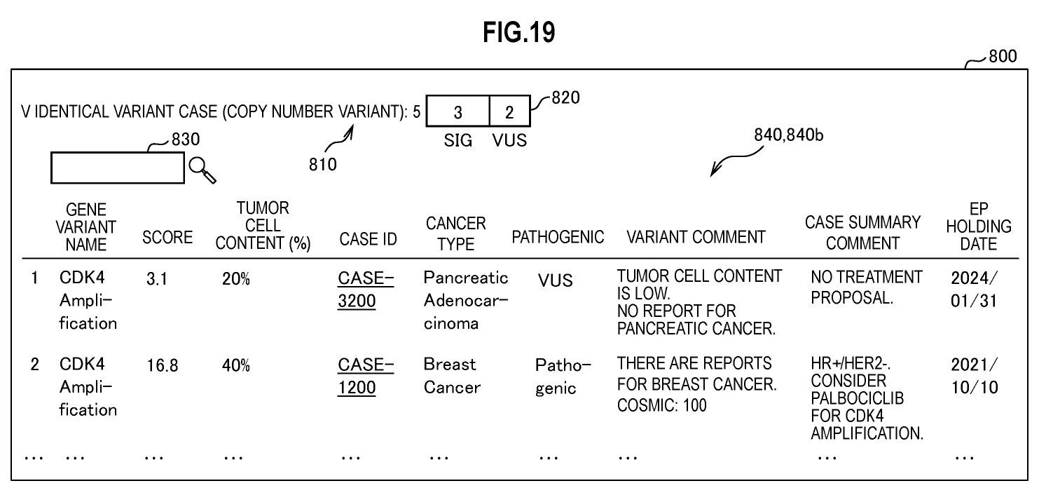

When the gene variant selected in the variant name display portion 713 in FIG. 17 is a copy number variant, a gene variant name, a score, and a tumor cell content are displayed in an identical variant information display portion 840b as shown in FIG. 19. Further, the identical variant information display portion 840b displays a case ID, a cancer type, a pathogenic flag, a variant comment, a case summary comment, an expert panel (EP) holding date, and the like. The score is a CNA score stored in the copy number variant table 212 in FIG. 11.

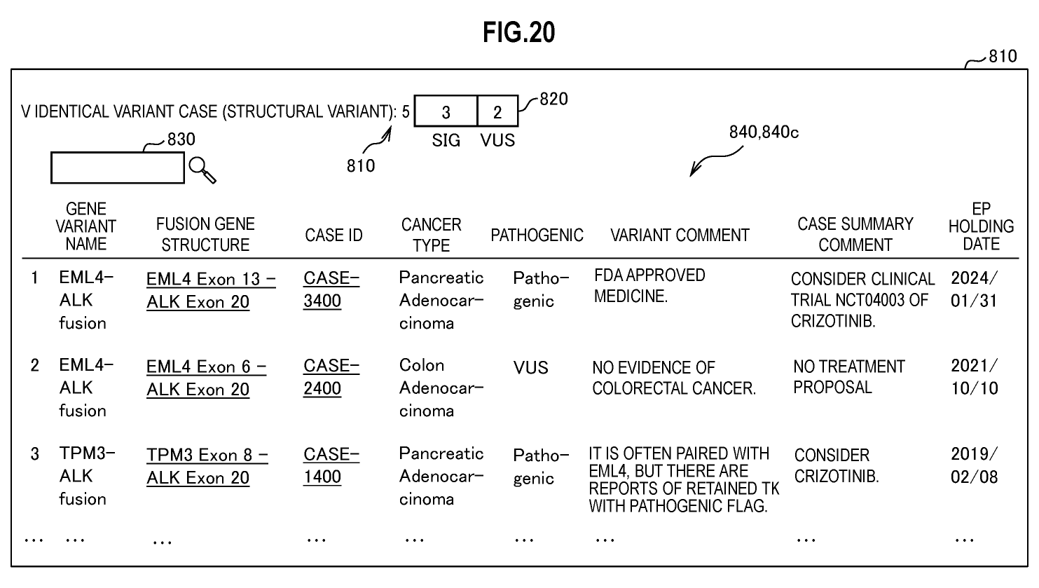

When the selected gene variant is a structural variant, a gene variant name, a fusion gene structure, a case ID, a cancer type, and a pathogenic flag are displayed in an identical variant information display portion 840c as shown in FIG. 20. Further, the identical variant information display portion 840c displays a variant comment, a case summary comment, an expert panel (EP) holding date, and the like.

In the example shown in FIG. 19, display is performed when the selected gene variant is a structural variant in a case where two genes are fused. In the case of such fusion, in the structural variant table 213 shown in FIG. 11, a gene variant in which both genes to be fused match the selected gene variant is displayed. When there is no gene variant in which both genes to be fused match the selected gene variant, a gene variant in which one of a cancer gene or a cancer suppression gene matches the gene variant selected in the variant name display portion 713 in FIG. 17 is displayed.

Gene variants displayed in the identical variant information display portion 840 are displayed in the order sorted in step S760 in FIG. 14. Accordingly, a latest interpretation result among a plurality of interpretation results for a gene variant related to a patient subjected to a previous test is preferentially displayed in the identical variant information display portion 840. That is, a plurality of interpretation results are displayed in order of expert panel holding dates. Accordingly, a user can refer to interpretation in ascending order of test dates for a gene variant that is identical with a gene variant detected in a current test.

A member of an expert panel can confirm the identical variant in a previous case for a target gene variant of the expert panel by displaying the identical variant information display portion 840.

In the identical variant information display portion 840a shown in FIG. 18, a name of a gene variant that is determined to be pathogenic and a name of a gene variant that cannot be distinguished whether the gene variant is pathogenic (VUS) may be displayed in different character colors. Similarly, in the identical variant information display portion 840b shown in FIG. 19, a name of a gene variant that is determined to be pathogenic and a name of a gene variant that cannot be distinguished whether the gene variant is pathogenic (VUS) may be displayed in different character colors. The same applies to the identical variant information display portion 840c shown in FIG. 20.

A variant interpretation technique using a prediction technique such as AI has been developed so far. However, since such a technique still has a problem in accuracy and only scores and classifications are often displayed as a test result instead of interpretation, it is difficult to say that the technique is effectively used in a preliminary study or an expert panel.

Depending on a test company, whether a variant is pathogenic is distinguished, or only detailed information is added when whether a variant is pathogenic cannot be distinguished, and whether a variant is pathogenic needs to be examined in a preliminary study or independently examined by an expert panel. Detailed added information differs depending on a test company.

When a study in an expert panel is performed based on reports created by test companies, results of preliminary studies are separately managed, and it is difficult to refer to the results in a unified manner. In addition, information of a preliminary examination result and information of a final determination result are not stored in a database, and it is difficult to say that the information is effectively used.

In the first embodiment, the number of interpretation results of identical gene variants examined in a previous expert panel is displayed in the identical variant case number display portion 810 in FIGS. 18 to 20. The identical gene variant refers to a gene variant that is identical with a gene variant to be processed.

The identical variant information display portion 840 displays contents of interpretation results in a previous expert panel. Then, a member examines the gene variant to be processed based on interpretation of the previous expert panel.

According to the processing shown in FIG. 15, correction is performed when a variant position or the like is different caused by a test or an analysis.

According to the first embodiment, since the identical variant case display portion 800 is displayed, a member can refer to a previous pathogenic interpretation result, so that the member can easily examine a gene variant. Accordingly, it can be expected to reduce the burden of a variant interpretation task in a preliminary study or on a member of an expert panel and prevent an interpretation error.

### Second Embodiment

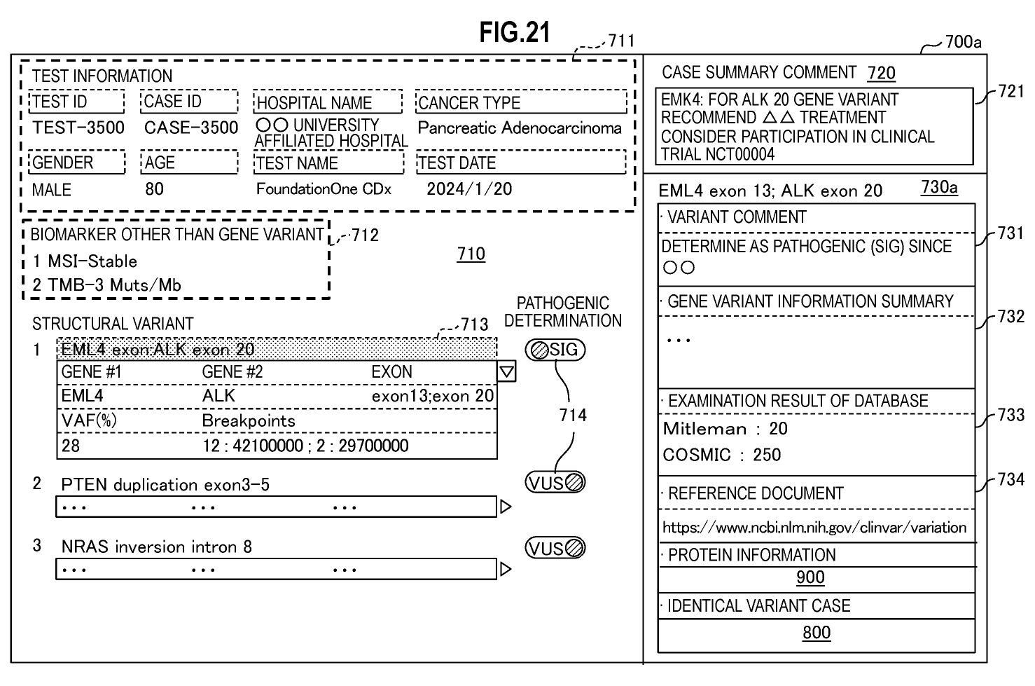

FIG. 21 is a diagram showing an example of an expert panel support screen 700a according to a second embodiment.

The expert panel support screen 700a shown in FIG. 21 is different from the expert panel support screen 700 shown in FIG. 17 in that a protein information display portion 900 is added to a variant detail display portion 730a. The other configurations are the same as those of the expert panel support screen 700 shown in FIG. 17, and thus description thereof will be omitted.

The protein information display portion 900 is displayed when the gene variant selected in the variant name display portion 713 is fusion among structural variants. Then, information on a protein generated by a gene in which a gene variant occurred is displayed.

### Protein Information Display Portion 900

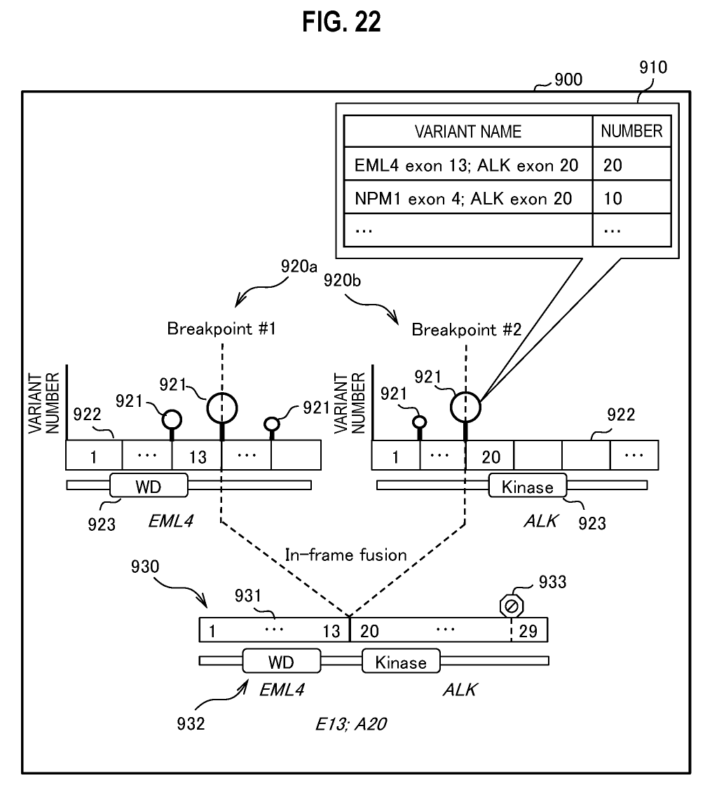

FIG. 22 is a diagram showing an example of the protein information display portion 900.

The protein information display portion 900 displays information on a protein related to a gene variant of a patient (first patient) subjected to a current test.

FIG. 22 shows an example in which the gene variant selected in the variant name display portion 713 in FIG. 21 is a structural variant in which two genes are fused. Hereinafter, the gene variant selected in the variant name display portion 713 in FIG. 21 is appropriately referred to as a "selected gene variant".

The protein information display portion 900 includes a breakpoint information display portion 910, pre-variant gene display portions 920a and 920b, and a variant gene display portion 930. The gene variant selected in the variant name display portion 713 in FIG. 21 is appropriately referred to as a "selected gene variant".

The pre-variant gene display portion 920b displays a gene ("ALK gene") located downstream of a fusion gene before fusion as a gene composition display portion 922. The gene composition display portion 922 displays a composition of the gene ("ALK gene" in the example shown in the pre-variant gene display portion 920b). Numbers shown in the gene composition display portion 922 are numbers of exons.

A protein code display portion 923 displays a code position of a protein generated by a gene displayed in the gene composition display portion 922. For example, in the example shown in the pre-variant gene display portion 920b, Kinase domain is encoded after the exon number "20".

A breakpoint of a gene is displayed in a breakpoint display portion 921. The break point is a cleaved point of chromosome (DNA). DNA is cleaved at the break point, and deletion, translocation, and insertion occur. In the breakpoint display portion 921, a frequency of a target breakpoint is displayed as a lollipop plot. The frequency is, for example, the number of documents in which the target breakpoint is described. The frequency is information obtained by the display processing unit 101 searching the external database 62 such as UniProt (registered trademark).

When a user clicks the breakpoint display portion 921, the breakpoint information display portion 910 is displayed. The breakpoint information display portion 910 displays a name (variant name) of a gene variant related to the clicked breakpoint, the number of documents in which the gene variant is described, and the like. The number of documents is information obtained by the display processing unit 101 searching the external database 62 such as UniProt (registered trademark).

The variant gene display portion 930 displays information related to a chimeric protein that may be generated by fusion variant. Specifically, a chimeric transcription product (reference numeral 931) after fusion and a protein structure (reference numeral 932) generated by the chimeric transcription product are displayed. An exon number is displayed in the transcription product.

The variant gene display portion may display prediction information such as an open reading frame (ORF) and in-frame including a position of a stop codon (reference numeral 933).

By displaying the protein information display portion 900, a structure of a protein (chimeric protein) estimated to be generated by gene fusion is visualized, which can facilitate understanding for a user.

Information displayed in the protein information display portion 900 is acquired by the display processing unit 101 referring to the external database 62.

The invention is not limited to the embodiments described above and includes various modifications. For example, the embodiments described above have been described in detail in order to facilitate understanding of the invention, and the invention is not necessarily limited to those including all the configurations described above. A part of a configuration of a certain embodiment can be replaced with a configuration of another embodiment, and a configuration of another embodiment can be added to a configuration of a certain embodiment. In addition, another configuration can be added to, deleted from, or replaced with a part of a configuration of each embodiment.

Although a personal computer (PC) is assumed as the terminal 5 in the present embodiment, a smartphone or a tablet terminal may be used as the terminal 5.

Some or all of configurations, functions, the display processing unit 101 to the communication processing unit 105, the interpretation database 2, and the like described above may be implemented by hardware, for example, by designing an integrated circuit. As shown in FIG. 2, the configurations, functions, and the like described above may be implemented by software by a processor (calculation device 122) such as a CPU interpreting and executing a program that implements each function. Information such as a program, a table, or a file that implements each function can be stored in an HD, or can be stored in a recording device such as the memory 121 and an SSD, or a recording medium such as an integrated circuit (IC) card, a secure digital (SD) card, or a digital versatile disc (DVD).

Control lines and information lines considered to be necessary for description are shown in each embodiment, and not all control lines and information lines in a product are necessarily shown. Actually, it may be considered that almost all the configurations are connected to one another.

What is claimed is:

1. An expert panel support system comprising:
an acquisition unit configured to acquire interpretation result data that is data of an interpretation result for each gene variant of a first patient subjected to a gene panel test; and
a display processing unit configured to display, based on the interpretation result data, a result of a test performed on the first patient for each gene variant on a terminal used by a participant of an expert panel, wherein
the display processing unit displays, based on the interpretation result data, interpretation results of a gene variant of a plurality of second patients, who are patients different from the first patient and previously subjected to the gene panel test before the gene panel test performed on the first patient, on the terminal in association with the interpretation result of the gene variant of the first patient.

2. The expert panel support system according to claim 1, wherein
when the interpretation result of the gene variant of the first patient is displayed in association with the interpretation results of the gene variant of the second patients, the display processing unit displays information related to the number of gene variants specified for the second patients on the terminal based on the interpretation result data.

3. The expert panel support system according to claim 1, wherein
when the interpretation result of the gene variant of the first patient is displayed in association with the interpretation result of the gene variant of the second patients, a latest interpretation result among a plurality of interpretation results for the gene variant of the second patients is preferentially displayed.

4. The expert panel support system according to claim 1, wherein
the number of cases in which the interpretation result is determined to be pathogenic and the number of cases in which an otherwise determination is made are displayed on the terminal in a distinguished manner for the interpretation results of the second patients.

5. The expert panel support system according to claim 1, wherein
gene variants that are substantially identical gene variants but are expressed by different names and variant positions between tests are displayed as identical variants on the terminal.

6. The expert panel support system according to claim 1, wherein
information on a protein related to the gene variant of the first patient is displayed on the terminal.

7. An expert panel support method comprising: a processing device that displays an expert panel support screen for use in an expert panel and an expert panel preliminary study executing:
an acquisition step of acquiring interpretation result data that is data of an interpretation result for each gene variant of a first patient subjected to a gene panel test; and
a display processing step of displaying, based on the interpretation result data, a result of a test performed on the first patient for each gene variant on a terminal used by a participant of the expert panel, wherein
in the display processing step, the processing device displays, based on the interpretation result data, interpretation results of a gene variant of a plurality of second patients, who are patients different from the first patient and previously subjected to the gene panel test before the gene panel test performed on the first patient, on the terminal in association with the interpretation result of the gene variant of the first patient.
